(19) United States
(12) Patent Application Publication
(10) Pub. No.: US 2004/0161443 A1
(43) Pub. Date: Aug. 19, 2004

(54) Vehicle

(76) Inventors: Sheila MacNeil, Sheffield (GB); Rob Short, Sheffield (GB); Chris Hunter, Sheffield (GB); John Haycock, Sheffield (GB); Nick Williams, Sheffield (GB); Anthony J R Ryan, Sheffield (GB)

Correspondence Address: MYERS BIGEL SIBLEY & SAJOVEC, PO BOX 37428, RALEIGH, NC, 27627, US

(21) Appl. No.: 10/474,994

(22) PCT Filed: Apr. 17, 2002
(86) PCT No.: PCT/GB02/01713

(30) Foreign Application Priority Data
Apr. 17, 2001 (GB) 0109348.3
Apr. 17, 2001 (GB) 0109347.5

(51) Int. Cl. A61K 38/24

(52) U.S. Cl. 424/423; 514/12

(57) ABSTRACT

The present invention provides a vehicle for use in tissue engineering and/or surgical procedures comprising a Melanocyte Stimulating Hormone (MSH) receptor ligand: The vehicle may be a prosthesis, implant, matrix, stent, gauze, bandage, plaster, biodegradable matrix, or polymeric film the invention also provides a method of forming a vehicle of the invention.

### FIELD OF THE INVENTION

The present invention relates to vehicles for use in therapeutic or cosmetic tissue engineering/surgical procedures comprising Melanocyte Stimulating Hormone (MSH) and to methods of coupling MSH for use in such vehicles.

### BACKGROUND OF THE INVENTION

The need for replacement body parts in combination with the shortage of donor tissue and/or organs has led to the production of tissue engineered products.

Tissue engineering is an emerging science which has implications with respect to many areas of clinical and cosmetic surgery. More particularly, tissue engineering relates to the replacement and/or restoration and/or repair of damaged and/or diseased tissues, for example for cosmetic purposes or to return the tissue and/or organ to a functional state. For example, and not by way of limitation, tissue engineering is useful in the provision of skin grafts to repair wounds occurring as a consequence of contusions, burns, or failure of tissue to heal due to venous or diabetic ulcers.

Tissue engineering is also practised during replacement of joints because of degenerative diseases such as arthritis, replacement of coronary arteries due to damage as a consequence of various environmental causes (e.g., smoking, diet) and/or congenital heart disease including replacement of arterial/heart valves, organ transplantation, repair of gastric ulcers, replacement of bone tissue to treat diseases such as osteoporosis, replacement of muscle and nerves as a consequence of neuromuscular disease or damage through injury and replacement of bladder materials to counter urological disease.

Unfortunately, the culturing of cell/tissues in vitro represents only part of the problem faced by tissue engineers. In many examples the growth of cells in culture is not the major obstacle to success. It is the transfer of the cells/tissue via a suitable vehicle so that the cells/tissue are incorporated into the patient to be treated which represents a further, more taxing problem. By way of example and not by way of limitation a suitable vehicle may include culture-ware, prostheses, implants, 3-dimensional matrix supports, extracelular matrix protein coated dressing, bandages or plasters.

Vehicles suitable for the transfer of replacement tissue have to satisfy certain requirements if they are to be useful in tissue engineering. Transfer vehicles typically have the following characteristics;

i) they provide a surface to which cells may become securely attached;

ii) they allow attached cells to grow and divide unhindered by the attachment surface;

iii) where appropriate, they provide an attachment surface which does not influence the differentiated (or undifferentiated) state of the attached cells;

iv) they maintain cells in a sterile and immunologically silent status;

v) they are minimally toxic to the patient;

vi) they do not transmit bacterial or viral disease; and

vii) they provide a surface from which attached cells may easily detach and subsequently invade the tissue site requiring replacement, restoration or repair.

Other surgical procedures rely upon vehicles which are not used in a cell transfer context and which may be substantially cell free. By way of example and by no means of limitation the vehicle may be a bandage or device to reduce inflammation and used in a wound healing context e.g., for bums injuries, venous leg ulcers, diabetic ulcers or in inflammatory skin diseases such as psoriasis or eczema.

MSH autocrine production by skin cells (keratinocytes, melanocytes and fibroblasts) is part of an intrinsic defence mechanism, assisting cells to survive periods of inflammation and oxidative stress.

MSH is a 13 amino acid peptide which is produced in the pituitary, gut and skin. It is best known for its role in the control of melanogenesis in pigmentary cells. Understanding of extra-pigmentary actions of MSH has developed rapidly in recent years. A number of studies suggest that visible pigmentation may only represent a small physiological role for MSH in skin. Previously only melanocytes were thought to respond to MSH. It now seems that the ability to respond to MSH is shared by a number of cells in skin, not just those able to produce a pigment. Furthermore, a number of different cell types such as melanocytes, cutaneous epithelial cells, bronchial epithelial cells, bladder epithelial cells, corneal epithelial cells, endothelial cells, fibroblasts, smooth muscle cells and monocytes possess the melanocortin-1 receptor (MC-1R) for MSH.

There is little doubt that tissue engineered approaches to wound repair will present significant therapeutic benefits compared with existing treatments. Several issues however are as yet currently unresolved. In particular there is a need to develop approaches to protect cells during the initial period of inflammation which occurs when tissue engineered materials are used clinically. The initial inflammatory response is thought to be responsible for the destruction and failure of many materials within the first few days of grafting. An adverse inflammatory response is also often observed when surgical devices such as coronary artery stents and prosthetic devices are used and even when autologous cells are reintroduced into the body.

### STATEMENTS OF THE INVENTION

According to the present invention there is provided a vehicle for use in tissue engineering/surgical procedures comprising a MSH receptor ligand.

The term vehicle may be defined as any structure or device for use in tissue engineering/surgical procedures. By way of example and not by way of limitation, the term includes a prosthesis, implant, matrix, stent, gauze, bandage, plaster, biodegradable matrix; or polymeric film.

Preferably the vehicle has minimal patient toxicity and does not elicit an unfavourable reaction when delivered to a patient.

The MSH receptor ligand is suitably MSH or another peptide comprising a functional fragment of MSH, for example it may be a functional fragment of MSH. Alternatively, the receptor ligand may be a peptide comprising a structural variant of MSH and having MSH receptor binding function. The term functional fragment includes any peptide derived from MSH (eg 6 and 3 amino acid fragments of MSH can also achieve the same biological effect).

The term structural variant includes sequence variants which retain the same biological activity, or have increased biological activity (eg a superpotent peptide exists which, like MSH, is 13 amino acids long). Table 1 lists the MSH full length sequences (of which the MSH full length sequence number 6 is a super potent peptide) and fragment sequences.

TABLE 1
MSH full length and fragment sequences
Three letter amino acid code used

| | |
|---|---|
| Alanine | Ala |
| Arginine | Arg |
| Asparigine | Asn |
| Aspartic acid | Asp |
| Asparagine | Asx |
| Cysteine | Cys |
| Glutamic acid | Glx |
| Norleucine | Nle |
| Glutamine | Glx |
| Glycine | Gly |
| Histidine | His |
| Isoleucine | Ile |
| Leucine | Leu |
| Lysine | Lys |
| Methionine | Met |
| Norleucine | Nle |
| Phenylalanine | Phe |
| Proline | Pro |
| Serine | Ser |
| Threonine | Thr |
| Tryptophan | Trp |
| Tyrosine | Tyr |
| Valine | Val |

L = Laevo (all amino acid conformations unless indicated)
D = Dextro (indicated where amino acid is not of L conformation)
Ac = Acetyl

MSH full-length sequences

| | |
|---|---|
| 1. α-MSH | Ac-Ser-Tyr-Ser-Met-Glu-His-Phe-Arg-Typ-Gly-Lys-Pro-Val-NH₂ |
| 2. α-MSH (free acid) | Ac-Ser-Tyr-Ser-Met-Glu-His-Phe-Arg-Typ-Gly-Lys-Pro-Val-OH |
| 3. (Des-acetyl)-α-MSH | H-Ser-Tyr-Ser-Met-Glu-His-Phe-Arg-Typ-Gly-Lys-Pro-Val-NH₂ |
| 4. (Diacetyl)-α-MSH | Ac-Ser(Ac)-Tyr-Ser-Met-Glu-His-Phe-Arg-Typ-Gly-Lys-Pro-Val-NH₂ |
| 5. (Nle₄)-α-MSH | Ac-Ser-Tyr-Ser-Nle-Glu-His-Phe-Arg-Typ-Gly-Lys-Pro-Val-NH₂ |
| 6. (Nle₄, D-Phe₇)-α-MSH | Ac-Ser-Tyr-Ser-Nle-Glu-His-D-Phe-Arg-Typ-Gly-Lys-Pro-Val-NH₂ |

MSH fragment sequences

| | |
|---|---|
| 7. (Ac-Nle₄, Gln₅, D-Phe₇, D-Trp₉)-α-MSH (4-10) | Ac-Nle-Gln-His-D-Phe-Arg-D-Trp-Gly-NH₂ |
| 8. (Ac-Cys₄, D-Phe₇, Cys₁₀)-α-MSH (4-13) | Ac-Cys-Glu-His-D-Phe-Arg-Trp-Cys-Lys-Pro-Val-NH₂ |
| 9. α-MSH (11-13) | H-Lys-Pro-Val-NH₂ |
| 10. α-MSH (11-13) free acid | H-Lys-Pro-Val-OH |
| 11. Acetyl-α-MSH (11-13) | Ac-Lys-Pro-Val-NH₂ |
| 12. Acetyl-(D-Lys₁₁, D-Val₁₃)-α-MSH (11-13) | Ac-D-Lys-Pro-D-Val-NH₂ |
| 13. Acetyl-(D-Val₁₃)-α-MSH (11-13) | Ac-Lys-Pro-D-Val-NH₂ |
| 14. α-MSH (10-13) | H-Gly-Lys-Pro-Val-OH |
| 15. α-MSH (10-13) | H-Gly-Lys-Pro-Val-NH₂ |
| 16. Acetyl-α-MSH (10-13) | Ac-Gly-Lys-Pro-Val-NH₂ |

The invention also includes vehicles comprising a peptidomimetic activity of any of the aforesaid peptides, i.e. materials that convey the same biological activity but do not necessarily have the same structure as these peptides.

In one embodiment of the invention, the vehicle comprises immobilised MSH. In an alternative embodiment the MSH is slowly released by proteolytic cleavage.

Proteolytic Cleavage and Release of MSH

A method for local delivery of MSH peptide fragments locally from a support biomaterial surface is suggested by incorporation of an endopeptidase/proteinase/proteolytic cleavage site proximal to the MSH peptide. Proteinase activity arising from the host tissue surrounding an implanted device would facilitate the enzyme-mediated cleavage and release of a bioactive MSH peptide fragment, thereby permitting subsequent receptor mediated interactions between MSH peptide and host tissue receptors.

A single proteolytic cleavage site proximal to the MSH peptide is suggested, however the amino acid sequence design for a given site is potentially large: a) due to the number of different proteolytic cleavage sites available for a given proteolytic enzyme and b) due to the number of tissue enzymes potentially able to act in this respect Therefore two examples are explained below to illustrate the design methodology: 1) for matrix metalloproteinase I (MMP1: fibroblast collagenase) and 2) for plasmin. (fibrin/fibrinogen cleavage). In each case the example MSH peptide fragment released is based on MSH 11-13 (Lys-Pro-Val) or MSH 10-13 (Gly-Lys-Pro-Val).

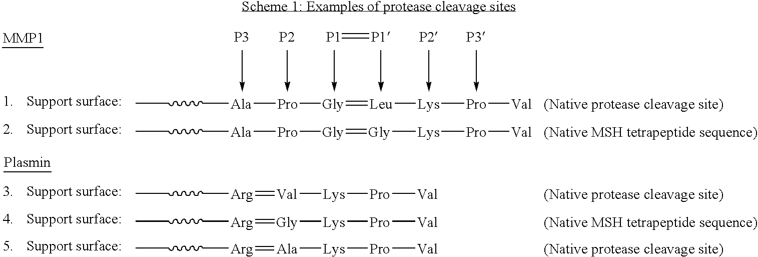

The protease cleavage site is indicated by =

The protease cleavage nomenclature (e.g. P1/P1′) is shown for sequence 1 above.

In the above scheme a bioactive MSH tetrapeptide is released. However any of the MSH peptide sequences (detailed in Table 1) would be candidate bioactive peptides for proteolytic release.

Where the protease cleavage site is indicated above as native (e.g MMP1, example 1) an MSH 10-13 sequence is released C-terminal to this. In this case the MSH 10 position amino acid is not native (as Gly is native), but a substituted amino acid with similar chemical properties is present in the P1′ position (e.g. Ala, Leu or Val—ie. hydophobicity maintained). Where a native MSH 10-13 tetrapeptide sequence is indicated above (e.g. MMP1, example 2) the cleavage site is not entirely native to the protease. Again, an amino acid with similar properties has been substituted into the P1 cleavage position (e.g. Ala/Val verses Gly, maintaining the hydrophobicity using a similar aliphatic side-chained amino acid). If cleaved specifically by MMP1, this would release the native MSH 10-13 peptide for subsequent interaction with the host tissue receptors.

This common generic design of protease cleavage sites linked to the MSH sequences will result in a large number of putative amino acid sequences to fulfill an MSH bioactive peptide release function. Therefore instead of listing an exhaustive number of cleavage site/MSH sequence combinations, a limited number of common tissue proteases are suggested which we expect to be relevant as candidate enzymes for potential ability to release adjacent MSH peptides. Individual designs for particular protease cleavage sequences linked to a particular MSH sequences would therefore exist for each protease/MSH peptide combination.

In one embodiment of the invention, MSH (or a structural or functional fragment thereof) is associated without concomitant cell attachment. In this instance, MSH peptides may be associated with bandages/dressings or beads for the treatment of acute or chronic inflammatory epithelial disorders. Thus applied to bandages or beads it could be used for the treatment of chronic ulcers (diabetic, non-healing venous or arterial ulcers or pressure sores), burns injuries (e.g. paediatric scalds) or inflammatory skin diseases (where excessive inflammation is viewed as being a contributory factor to the condition e.g. psoriasis and eczema). In these applications, it is envisaged that the bandage/dressing will be used to reduce the extent of the inflammation which may reasonably be expected to increase the rate of healing etc. Subsequent application of cells may/may not follow as part of a treatment to accelerate healing or achieve wound closure. MSH peptides immobilised on beads could be used for delivery to internal epithelial surfaces suffering from inflammation e.g. nasal mucosa (a treatment of hayfever) intestinal epithelia (for chronic inflammatory conditions such as irritable bowel syndrome, Crohns and Coeliac disease) or respiratory epithelial surfaces (for asthma). Alternatively MSH peptides may be associated with implantable materials or devices such as coronary artery stents, prostheses, heart valves or any device which is inserted into the body where reducing the ability of the device to cause inflammation would be desirable.

In an alternative embodiment of the invention, MSH peptides may also be associated with a bandage or dressing for concomitant cell attachment. This method may be applied to skin delivery devices for treatment of chronic ulcers and burns, possibly as a follow on from an application where MSH peptides are immobilised without concomitant cell attachment. The vehicle comprises a cell carrier surface to which a cell may become associated e.g., surfaces on which epithelial cells such as epidermal, keratinocytes, corneal epithelial cells, bladder epithelial cells or gut epithelial cells attach. A wide range of implantable tissue-engineered devices such as tissue engineered heart valves, reconstructed liver, bladder or coronary artery stents are coated in such a way as to promote endothelial cell attachment. Any of these could benefit from the inclusion of MSH peptides to assist cells on the devices (and adjacent cells) in their response to pro-inflammatory cytokines and oxidative stress.

Preferably a cell which becomes associated to the vehicle of the invention possesses the MC-IR receptor and attaches to the MSH receptor ligand.

In a yet further preferred embodiment of the invention said vehicle is suitable for use with cells of mammalian origin, and more preferably cells of human origin.

More preferably said cell is selected from cell types such as: keratinocytes; melanocytes, cutaneous epithelial cells, bronchial epithelial cells, bladder epithelial cells, corneal epithelial cells, endothelial cells, fibroblasts, smooth muscle cells, monocytes, gastrointestinal mucosal epithelial cells and oral mucosa epithelial cells.

It will be apparent to one skilled in the art, that the vehicle of the invention is useful in clinical applications where cells could be grown on surfaces of substrates prior to application to, for example and not by way of limitation, acute and/or chronic and/or minor and/or severe cutaneous wounds (including venous and diabetic ulcers); and/or cartilage repair; and/or bone repair, and/or muscle repair; and/or nerve repair; and/or connective tissue repair; and/or blood vessel repair; and/or bladder repair.

According to an alternative embodiment of the invention there is provided a cosmetic vehicle comprising a cell carrier surface characterised in that said surface is linked, coupled or associated with MSH receptor ligand, wherein said vehicle is adapted to be applied and/or implanted into a patient requiring cosmetic tissue engineering.

According to an alternative embodiment of the invention there is provided a therapeutic vehicle comprising a cell carrier surface characterised in that said surface is linked, coupled or associated with MSH receptor ligand, wherein the vehicle is adapted to be applied and/or implanted into a patient requiring therapeutic tissue engineering.

The invention provides a vehicle comprising an MSH receptor ligand. The introduction of MSH into a vehicle of the invention assists MSH receptor possessing cells within, or migrating over said vehicle or construct to withstand inflammatory damage and therefore provides significant advantages over existing tissue engineering/ surgical vehicles.

The initial period of inflammation which occurs when tissue engineered materials are used clinically is currently dealt with by the use of imunosuppressant drugs such as cyclosporin. Cyclosporin and other such steroids may be delivered systemically or topically and are associated with a severe dampening of the immune system which makes them unsuitable for long term delivery. Advantageously, MSH does not block the immune system and is suitable for long term delivery.

Preferably the association of MSH receptor ligand to a vehicle of the present invention is achieved by one, or any of a combination of:

i) coupling of MSH peptides to surfaces via linkers, for example, polyethelene glycol (PEG) linkers;

ii) the association of MSH peptides with calixarenes or calixarene treated surface;

iii) immobilisation of MSH peptides to a plasma polymerised surface.

i) MSH Linking Molecules

It is known that RGD motifs can be linked to PEG e.g. Drumheller et al, 1994. The Scheme 2 describes the linkage of MSH to PEG.

According to a further embodiment of the invention, there is provided an alternative method of preparing a surface to which MSH receptor ligand is capable of being associated with, comprising:

i) providing a linking agent and MSH receptor ligand;

ii) providing conditions suitable for linking said agent with MSH receptor ligand; and

iii) bringing the linked molecule in contact with a cell surface to be treated.

In a preferred method of the invention said linking agent is polyethylene glycol. Other linking agents are available and can be used for this purpose.

ii) Calixerenes and MSH (See Scheme 3)

Calixarenes are amphiphilic molecules whose general structure is that of a molecular bowl on legs with the rim of the bowl lined by hydroxyl groups and the legs consisting of long chain alkyl groups. A detailed review of the different types of calixarenes and their methods of manufacture is given in Bohmer, Angew. Chem. Int. Ed. Engl. 1995, 34, 713-745, the entire disclosure of which is incorporated herein by reference for all purposes.

It is known that the hydroxyl groups lining the rim of the bowl of calixarenes can bond strongly to hydrophilic substrates and that if the calixarene also has hydrophobic pendant legs this can impart a highly water repellent surface to the substrate, see WO 97/39077. Hydrophobic surfaces may be rendered hydrophilic by a number of means, including by the plasma polymerisation of a hydrophilic ‘monomer’.

We have worked primarily with resorcinarenes (X=H) and pyrogallenes (X=OH) where n=4. However, X can be varied more widely (such as CH₂Z or OCH₂Z), and n can also be 6 or 8. The pendant Y group is a long chain alkyl or perfluoroalkyl in our current compounds, but incorporation of functional groups such as double bonds, triple bonds, SR, OH or SH at the terminus of the chain can also be done.^{1,9} Y may also be a long polyethylene oxide chain.

The main advantages of this approach would be the simplicity and low loading of the treatment which means that it could be readily applied to bulk materials. The other advantage is that if the compounds do form an ordered monolayer on the surface, then the immobilised/adsorbed species will be in a more defined environment (which itself could be modified, to mimic a cell surface).

In a further embodiment of the invention there is provided a calixarene associated, coupled or linked to a MSH receptor ligand.

In a yet further embodiment of the invention there is provided a method for coupling or linking a calixerene to a MSH receptor ligand comprising:

In the first instance, the pendant chains Y will be functionalised at the terminal positions with OH as previously described. These OH groups will be converted to NH2 according to well established synthetic techniques. Simple treatment of material with a solution of calixarenes will provide an ordered, amine functionalised surface to the material.

Alternatively, prior coupling of the calixarene and MSH can be undertaken using the coupling technology described earlier, and the whole construct used for material treatment.

Similarly, the calixarenes will be functionalised with a single MSH via a tether. Monofunctionalised calixarenes can be synthesised as described by S. Saito, D. M. Rudkevich and J. Rebek Jr, Org. Lett. 1999, 1 (8), 1241-1244 and converted to an amino functionalised form which will allow facile derivation with MSH tethered to polyethylene glycol.

The synthetic approaches would be to make the resorcinarenes in scheme shown below. The hydroxyl tailed compound is well known and has been synthesised by us, as is the alkene appended calixarene.

Fully functionalisable calixarenes which can be attached to tether either before or after calixarene binding to surface. That is, the NH2 forms is linked through an amide attachment to polyethylene glycol which has been appended with MSH. This is the same technology as for the other approaches.

In both cases, the level of surface functionalisation can be controlled by diluting the functionalised calixarene with calixarenes which have non-functional Y groups. Polyfunctional calixarenes (i.e. with 4 attachment sites per calixarene) see scheme 4.

iii) Plasma Polymerisation

Plasma polymerisation is a technique which allows an ultrathin (eg ca.200 nm) cross linked polymeric film to be deposited on a substrate of complex geometry and with controllable chemical functionality. As a consequence, the surface chemistry of materials can be modified, without affecting the bulk properties of the substrate so treated.

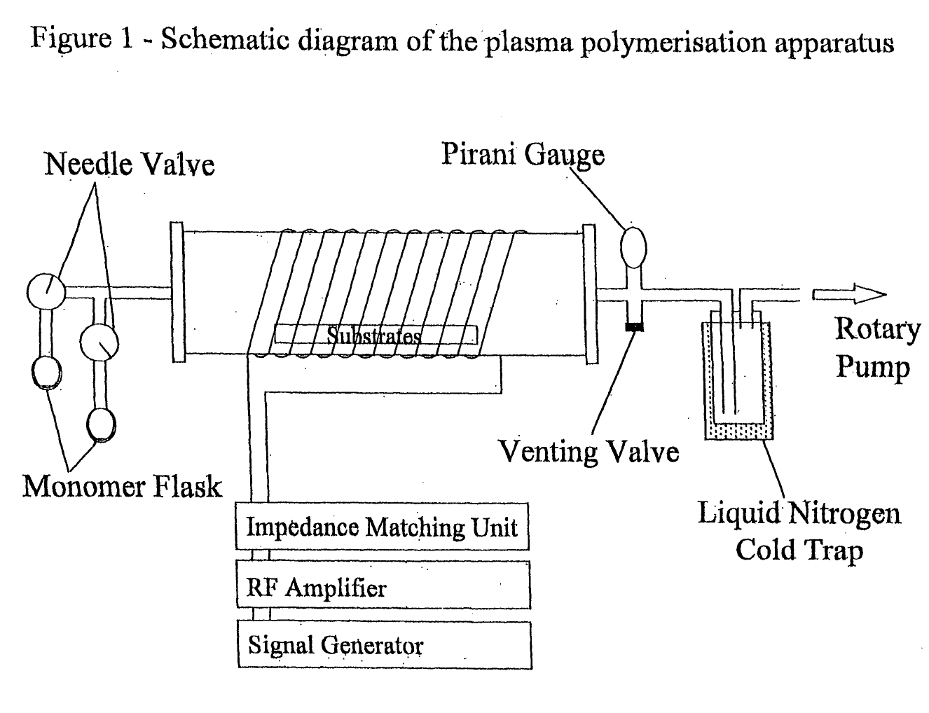

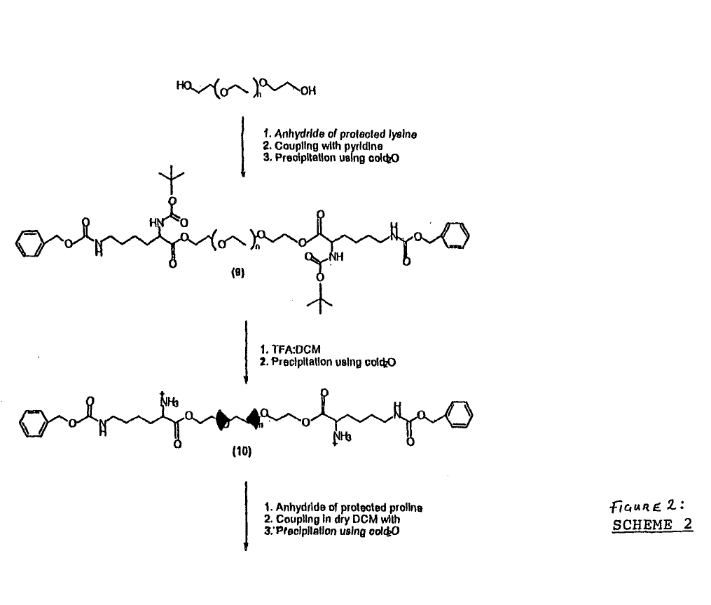

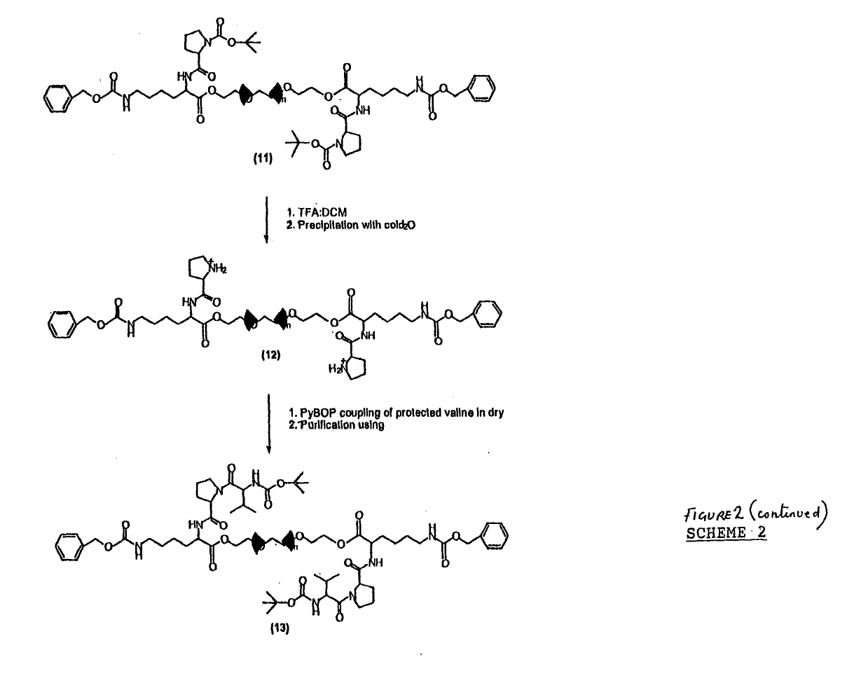

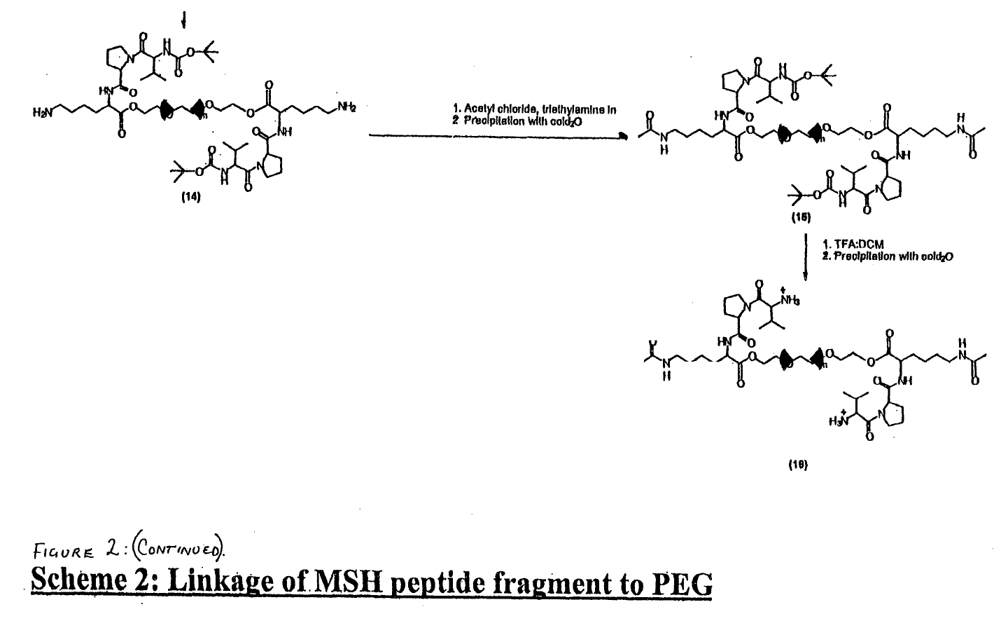

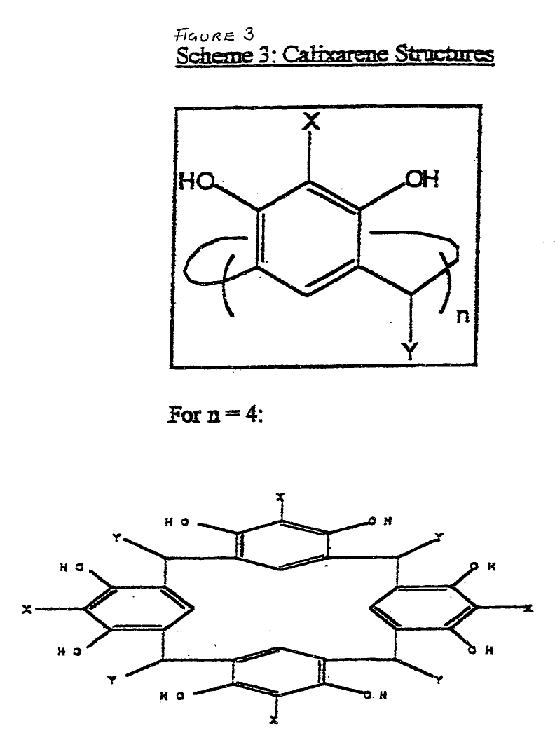

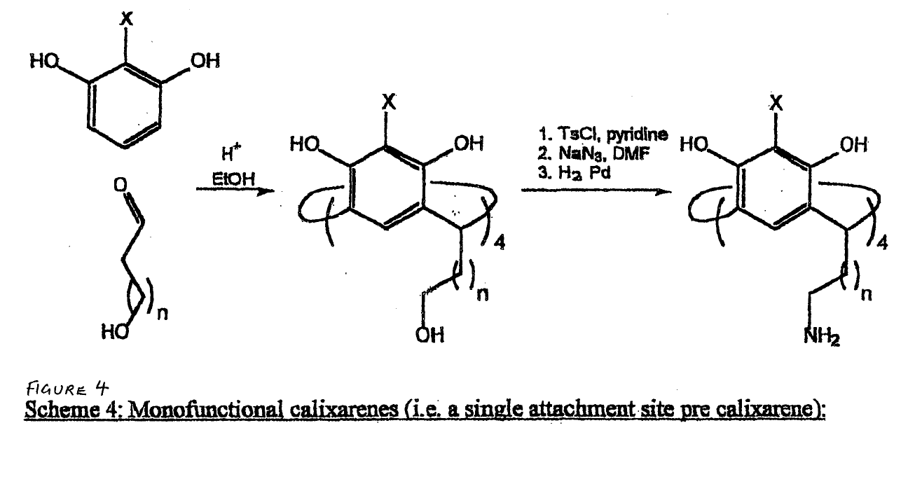

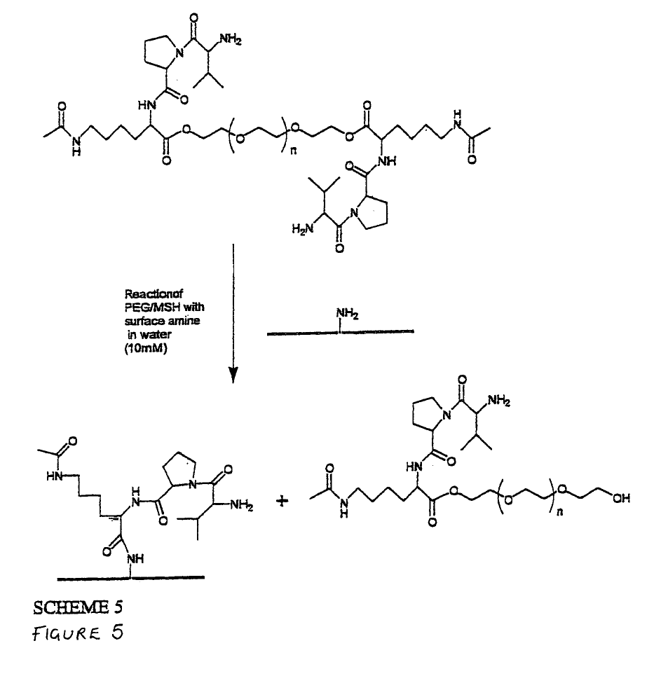

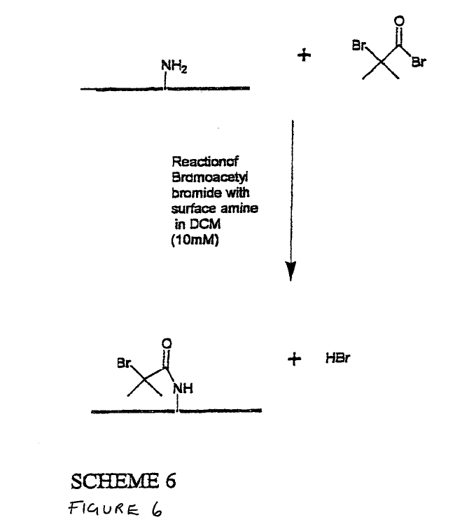

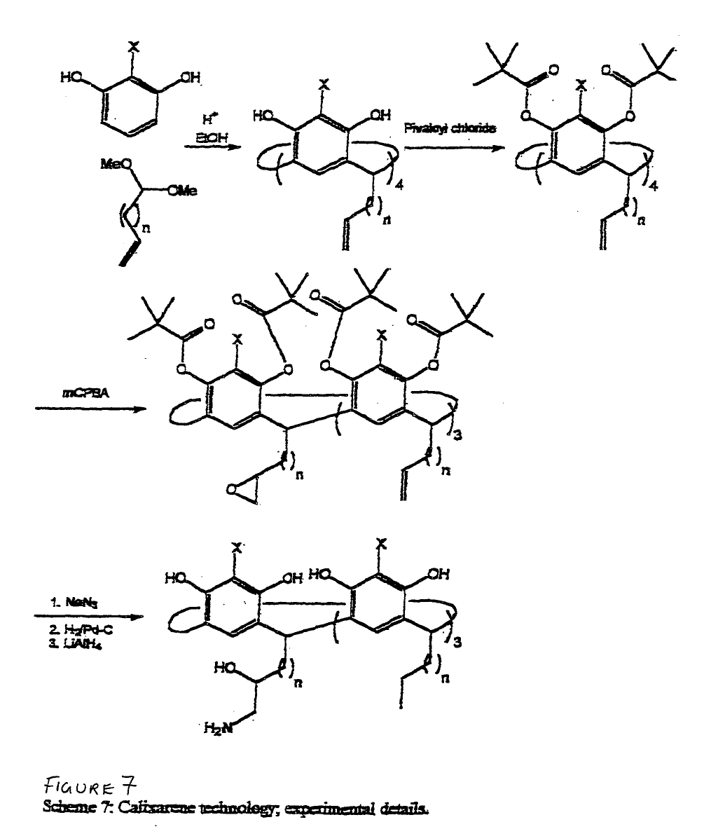

Plasmas or ionised gases are commonly excited by means of an electric field. They are highly reactive chemical environments comprising ions, electrons, neutrals (radicals, metastables, ground and excited state species) and electromagnetic radiation. At reduced pressure, a regime may be achieved where the temperature of the electrons differs substantially from that of the ions and neutrals. Such plasmas are referred to as “cold” or “non-equilibrium” plasmas. In such an environment many volatile organic compounds (eg volatile alcohols, volatile acids, volatile amines, or volatile hydrocarbons) neat or with other gases, eg Ar, have been shown to polymerise (H. K. Yasuda, Plasma Polymerisation, Academic Press, London 1985) coating both surfaces in contact with the plasma and those downstream of the discharge. The organic compound is often referred to as the “monomer”. The deposit is often referred to as “plasma polymer”. Plasma may be created and sustained by the application of an electric field to a gas (monomer) of reduced pressure. A wide range of plasma reactor geometries have been described, and means of power input (microwaves, radiofrequency, audio etc.) Herein we describe the use of an inductively coupled (13.56 MHz) RF plasma, but the numbers/values given for power input, gas pressure flow etc. may be readily adapted to other plasma reactors/power sources by those skilled in the art, please see FIG. 1.

Thin polymeric films can be obtained from the plasmas of volatile organic compounds (at reduced pressure of 10⁻² mbar and ideally less than 100° C.). In plasma polymer deposition, there is generally extensive fragmentation of the starting compound or ionised gas and a wide range of the resultant fragments or functional groups are undesirably incorporated into the deposit. The advantages of such a mode of polymerisation potentially include: ultra-thin pin-hole free film deposition; plasma polymers can be deposited onto a wide range of substrates; the process is solvent free and the plasma polymer is free of contamination. By employing a low plasma input power (low plasma power/monomer flow rate ratio) it is possible to fabricate films with a high degree of functional group retention. An example of such a low power/rate ratio is 2W and a flow rate of 2.0 sccm. A typical range would be 1-10W, and 1-5 SCCM). This is important where ‘retention’ of the molecular structure and chemical functionality of the deposit is required.

Other relatively low ratios may be used and are known to those skilled in the art. In the instance where a pulse wave is used corresponding corrections are made to the plasma power and flow rate, as is known by those skilled in the art. It will also be apparent to one skilled in the art that reactor conditions will vary depending on reactor geometry.

Alternatively, plasma polymer deposits may be formed by pulsing the plasmas or ionised gases. Plasmas are formed either from single monomer species or a combination of organic molecules. The coating of surfaces by plasma polymerisation is disclosed in PCT application WO00/78928.

For those instances without subsequent cell attachment, amine-containing compounds (primary, secondary or tertiary amine, with or without unsaturation) can be polymerised (or copolymerised with another molecule) to provide stable plasma polymerised amine platforms onto which MSH can be linked.

For the homopolymerisation of amines, primary, secondary or tertiary amine, with or without unsaturation (e.g. allyl amine) can be polymerised (under a fairly wide range of conditions) to produce a plasma polymerised platform onto which MSH can be linked. MSH peptides are tethered to a ‘linker’ molecule (e.g. PEG). This molecule will contain an active site (moiety) for the covalent linkage of the MSH peptide.

Copolymerisation is described in A. J. Beck, 1996. A preferred method is the plasma copolymerisation of an ethylene oxide (EO)-like molecule (e.g. triglyme or tetragyme) with a small amount of amine-containing compound (e.g., any of those identified above in homopolymerisation).

This strategy provides a plasma polymerised ‘EO-like’ platform with a controlled density of ‘reactive’ amine sites for the subsequent linking of the MSH fragment. The plasma polymerised EO-like platform confers general protein-resistant properties (S. Beyer et al, 1997, Y. J. Yu et al, 2000 and G. P. Lopez et al, 1992). This arises from the EO-character and reduces the extent of non specific protein adsorption keeping the associated MSH active for longer.

According to a further aspect of the invention there is provided a method of preparing a cell culture surface comprising:

i) providing at least one organic monomer;

ii) creating a plasma of said organic monomer; and

iii) coating the surface with said plasma to provide a cell culture surface to which MSH is capable of being associated.

In a preferred method of the invention said organic monomer is selected from the following:

a) Amines from allyl amine, butyl amine, heptyl amine, propyl amine etc. Candidate amines would be primary, secondary or tertiary with sufficient vapour pressures below 100 degrees C. (i.e. above 6.6 Pa at RTP, preferably about 130 Pa).

b) EO-type surfaces would be selected from tetraethylene glycol, dimethyl ether (tetraglyme), tetraethylene glycol divinyl ether, diethylene oxide divinyl ether and triethylene oxide monoallyl ether.

In one embodiment of the invention there is provided a vehicle for use in tissue engineering/surgical procedures comprising a Melanocyte Stimulating Hormone (MSH) receptor ligand wherein said vehicle has integral therewith, or applied thereto, a cell carrier surface obtainable by plasma polymerisation.

In a further embodiment of the invention, there is provided a method of treatment comprising administering to a patient an MSH receptor ligand in tissue engineering/surgical procedures.

In a further embodiment of the invention, there is provided MSH receptor ligand for use in skin reconstruction, bladder reconstruction, corneal epithelial grafts, coating of stents for coronary heart disease to prevent in-stent restenosis, contact lens coating, hip replacement or heart valve coatings.

Preferably the MSH receptor ligand is associated with a vehicle, preferably the vehicle comprises a cell carrier surface.

The association may by achieved by any appropriate means. Preferably the MSH receptor ligand is linked to the vehicle via linkers, especially polyethelene glycol (PEG) linkers, incorporation of MSH using calixarene, or by plasma polymerisation and coating with MSH.

In a further embodiment of the invention, there is provided a method of treatment comprising administering to a patient in need of therapeutic or cosmetic surgery, a cell carrier surface which is associated with MSH receptor ligand.

The invention will now be described by way of example and with reference to the following tables and figures.

### DETAILED DESCRIPTION OF THE INVENTION

Materials and Methods

Cells to be cultured on immobilised MSH will be epithelial, endothelial and neural crest derived cells, thus cutaneous epidermal keratinocytes, naso-gastro epithelial cells, intestinal epithelial cells, bronchial epithelial cells, corneal epithelial cells, bladder epithelial cells, embryonic stem cells, embryonic germ cells, haemopoietic stem cells, neural stem cells, osteoblasts, osteoclasts. For culture of cutaneous epidermal keratinocytes details are given in full in Chakrabarty et al, 1999, other epithelial, endothelial cells and neural crest cells will be cultured using established culture methodologies as published the in scientific literature.

Immobilisation or Adsorption Technology

A number of different approaches can be used for the linkage, coupling or association of MSH receptor ligand on carrier surfaces used for tissue engineering or other such tissue engineering devices.

(i) Peptide Linkage to PEG

As described in Scheme 2.

This scheme can be readily adapted to insert proteolytically cleavable moeties. A number of these are identified as in Scheme 1.

(ii) Synthesis of Hydroxy-Functionalised Calixarene (X=H, Y=(CH2)10OH):

Concentrated hydrochloric acid (3.2 ml) was added dropwise to a stirred solution of resorcinol (1.97 g, 17.9 mmol) and 1,1limethoxy-11-undecanol (4.15 g, 17.9 mmol) in ethanol (30 ml) at 0° C. The reaction mixture was heated at 55° C. for 18 hours under argon. After cooling, the yellow coloured solution was poured into water (250 ml) to yield a pale yellow precipitate. This was collected by filtration, washed with warm water (6×100 ml) and dried to give resorcarene as a pale yellow solid (4.58 g, 92%), m.p. 233-239° C. The crude material was recrystallised from methanol/chloroform (3.87 g, 78%) m.p. 237.5-238.5° C. Characterisation available.

Synthesis of Ene-Functionalised Calixarene (X=H, Y=(CH2)8CH=CH2):

Concentrated hydrochloric acid (3.2 ml) was added dropwise to a stirred solution of resorcinol (1.97 g, 17.9 mmol) and 10-undecenal (3.05 g, 17.9 mmol) in ethanol (30 ml) at 0° C. The reaction mixture was heated at 55° C. for 18 hours under argon. After cooling, the yellow coloured solution was poured into water (250 ml) to yield a pale yellow precipitate. This was collected by filtration, washed with warm water (6×100 ml) and dried to give resorcarene as a pale yellow solid (4.5 g, 90%). Characterisation available.

See scheme 7.

(iii) Linkage of Peptides to Plasmas Co-Polymer Surfaces Plasma Polymerisation

Summary of Experimental Conditions for Production of Ethylene Oxide ‘EO’-like Plasma Polymer and Co-polymerisation with Allylamine.

Monomers that will be used for the production of ‘EO-like’ PPs are tetraethylene glycol dimethyl ether (tetraglyme) or tetraethylene glycol divinyl ether.

In order to achieve a suitable flow rate of 2-3 sccm, monomers are heated in a water bath to 80-90° C. This yields a pressure of approximately 4-6×10⁻² mbar in the plasma reactor. A plasma power of 2-3 W and polymerisation time of 20 minutes are employed. Using these conditions, initial XPS results of a PP (tetraethylene gylcol dimethyl ether) have shown O/C ratios of 0.56 and a % retention of the ether functionality of ˜72% (from curve fitting the C is core level). This compares very favourably with an O/C ratio of 0.4-0.48 reported by Lopez et al, (1992) (N.B. no C 1s curve fits were presented)

Other monomers which will be of interest, due to their increased volatility and hence easier control of monomer flow are diethylene oxide divinyl ether and triethylene oxide monoallyl ether. The production of plasma polymers from these materials has been the subject of a recent study by Yu et al (2000) and Beyer et al (1997).

All of the above monomers may be co-polymerised with allylamine to provide reactive amine sites for MSH/peptide immobilisation. Copolymerisation is as described previously by Beck et al (1996).

PEG/MSH synthesis is as already described (Scheme 2). Scheme 5 illustrates the displacement reaction of a surface amine with the PEG/peptide molecule using MSH as an example.

Some initial results relating to the feasibility of this reaction are now presented. Bromoacetyl bromide undergoes a similar reaction with an amine as shown in Scheme 6. This reaction has been used to test the availability of surface amines for the reaction proposed in Scheme 5.

The feasibility of this scheme has been demonstrated using bromoacetyl bromide. The results of reaction of bromoacetyl bromide with surface amines in a PP of allylamine has been evaluated. The results are summarised in Tables 2 and 3.

TABLE 2
Summary of XPS results for washing and reaction of bromoacetyl
bromide with Allylamine PCPs (all samples prepared within 2 days
of plasma polymerisation).

| Sample | N/C | O/C | Br/N |
|---|---|---|---|
| Allylamine PP (01RF01) | 0.20 | 0.04 | |
| + dichloromethane (DCM) 5 mins | 0.19 | 0.06 | |
| + bromoacetyl bromide (10 mM in DCM), | 0.18 | 0.07 | 0.22 |
| DCM 5 mins | | | |
| + bromoacetyl bromide (10 mM in DCM), | 0.14 | 0.09 | 0.06 |
| DCM 5 mins, H₂O wash (2 × 3 min) | | | |

TABLE 3
Relative contributions of ‘physisorbed’ and ‘immobilised’ bromine
from peak fit of Br 3d core level (all samples prepared within 2 days of
plasma polymerisation). Charge corrected on C ls @ 285 eV.

| | ‘Phyisorb’ | ‘Imm.’ Br⁻ |
|---|---|---|
| Sample | Br⁻ (eV) | (eV) |
| + bromoacetyl bromide (10 mM in DCM), | 61% (68.0) | 39% (70.7) |
| DCM 5 mins | | |
| + bromoacetyl bromide (10 mM in DCM), | 39% (67.8) | 61% (70.7) |
| DCM 5 mins, H₂O wash (2 × 3 min) | | |

Upon reaction of the PP with bromoacetyl bromide, bromine is detected by XPS on the surface of the PP (Br/N=0.22). The results indicate that the reaction has taken place although care must be taken to distinguish between covalently immobilised bromine and that which is physisorbed to the substrate (N.B. we expect a strong charge-based interaction of Br⁻ with the allylamine PP). The data in Table 2 illustrate this point, with a change in the ratio of immobilised to adsorbed bromine upon washing with water. While presently the adsorbed bromine cannot be fully removed from the surface upon washing, the peak fit of the Br 3d core level allows one to distinguish the relative amount of this.

In addition to the results shown above, we have demonstrated the reaction of PEG/Cystine with a PP of allylamine, which results in the linking of cystine residues upon the surface (data not shown).

References:

1. A J Beck, R F Jones, R D Short, Polymer 1996, 24 (37) 5537-5539, Plasma co-polymerisation as a route to the fabrication of new surface chemistries with controlled amounts of specific chemical functionality

2. S Beyer, W Knoll, H Ringsdorf, J Hann Wang, R B Timmons, P Sluka. J Biomed Mater Res, 1997, 36, 181. ‘Reduced protein adsorption on plastics via direct deposition of triethylene glycol monoallyl ether’

3. Chakrabarty K H, Dawson R A, Harris P, Layton C, Babu M, Gould L, Phillips J, Leigh I, Green C, Freedlander E and Mac Neil S. (1999) Development of autologous human epidermal/dermal composites based on sterilised human allodermis for clinical use. Brit J Dermatol. 141, 811-823.

4. Drumheller P D, Ebert D L, Hubbell J A. Multifinctional poly(ethylene glycol) semi-interpenetrating polymer networks at highly selective adhesive substrates for bioadhesive peptide grafting. Biotechnology and Bioengineering 1994; 43:772-780.

5 Haycock J W, Rowe S J, Cartledge S, Wyatt A, Ghanem G, Morandini R, Rennie I G and Mac Neil S, 2000. α-melanocyte stimulating hormone reduces impact of proinflammatory cytokine and peroxide generated oxidative stress on keratinocyte and melanoma cell lines. Journal of Biological Chemistry, 275, 15629-15636.

6. Haycock J W, Wagner M, Moranbdini R, Ghanem G, Rennie I G and Mac Neil S. (1999) α-melanocyte stimulating hormone inhibits NF-κB activation in human melanocytes and melanoma cells. Journal of Investigative Dermatology 113:560-566

7. Hedley S J, Gawkrodger D J, Weetman A P, Morandini R, R Boeynaems J M, Ghanem G and Mac Neil S. (1998) Potential immunomodulatory role for a-MSH in normal human melanocytes and in melanoma cells. British Journal of Dermatology 138: 536-543.

8. Ichii-Jones Fm Lear J T, Heagerty A H M, Smith A G, Hutchinson P E, Osborne J, Bowers B, Jones P W, Davies E, Ollier W E R, Thomson W, Yengi L, Bath J, Fryer A A and Strange R C. (1998) Susceptibility to melanoma: influence of skin type and polymorphism in the melanocyte stimulating hormone receptor gene. Journal of Investigative Dermatology 111: 218-221.

9. G P Lopez, B D Ratner, C D Tidwell, C L Haycox, R J Rapoza and T A Horbett, J Biomed Mater Res, 1992, 26, 415. ‘Glow discharge plasma deposition of tetraethylene glycol dimethyl ether for fouling resistant biomaterial surfaces.

10. Morandini R, Boeynaems J M, Hedley S J, Mac Neil S and Ghanem G. (1998) Modulation of ICAM-1 expression by α-MSH in human melanoma cells and melanocytes. Journal of Cell Physiology 175: 276-282.

11. Y J Yu, R B Timmons, J S Jen and F E Molock. Coll Surf B, 2000, 18, 235. ‘Non fouling surfaces produced by gas phase pulsed plasma polymerisation of an ultra low molecular weight ethylene oxide containing monomer’

1. A vehicle for use in tissue engineering and/or surgical procedures comprising a Melanocyte Stimulating Hormone (MSH) receptor ligand.

2. A vehicle according to claim 1 wherein the vehicle is a prosthesis, implant, matrix, stent, gauze, bandage, plaster, biodegradable matrix, or polymeric film.

3. A vehicle according to claim 1 or claim 2 wherein the MSH receptor ligand is MSH, or a functional fragment thereof.

4. A vehicle according to claims 1 or claim 2 wherein the receptor ligand is a peptide comprising a structural variant of MSH and having MSH receptor binding function.

5. A vehicle according to any of the preceding claims wherein the MSH receptor ligand is immobilised.

6. A vehicle according to any of the preceding claims 1 to 4 wherein the MSH receptor ligand is released by proteolytic cleavage.

7. A vehicle according to claim 6 comprising a proteolytic cleavage site proximal to the MSH receptor ligand.

8. A vehicle according to any of the preceding claims wherein MSH receptor ligand is linked thereto by a linker.

9. A vehicle according to claim 8 wherein the linker is a polyethylene glycol (PEG) linker.

10. A vehicle according to any of the preceding claims further comprising a calixarene wherein the calixarene is associated, coupled or linked to the MSH receptor ligand.

11. A vehicle according to any of the preceding claims further comprising a plasma polymerised surface.

12. A vehicle according to any of the preceding claims for use in the treatment of acute or chronic inflammatory epithelial disorders.

13. A vehicle according to any of the preceding claims for use in the delivery of MSH receptor binding ligand to epithelial surfaces.

14. A vehicle according to any of the preceding claims further comprising a cell carrier surface to which a cell may become associated.

15. A vehicle according to claim 14 wherein the cell carrier surface is suitable for use with any or a combination of keratinocytes; melanocytes, cutaneous epithelial cells, bronchial epithelial cells, bladder epithelial cells, corneal epithelial cells, endothelial cells, fibroblasts, smooth muscle cells, monocytes, gastrointestinal mucosal epithelial cells and oral mucosa epithelial cells.

16. A vehicle according to any of the preceding claims for use in cartilage repair; bone repair; muscle repair; nerve repair, connective tissue repair; blood vessel repair; bladder repair.

17. A vehicle according to any of the preceding claims wherein the vehicle is adapted to be applied and/or implanted into a patient requiring cosmetic tissue engineering.

18. A vehicle according to any of the preceding claims wherein the vehicle is adapted to be applied and/or implanted into a patient requiring therapeutic tissue engineering.

19. A method of forming a vehicle of any of claims 1 to 18 comprising one, or any combination of the following steps:
i) coupling an MSH peptide to a surface via a linker,
ii) associating an MSH peptide with a calixarene or a calixarene treated surface;
iii) immobilisation of MSH peptides to a plasma polymerised surface.

20. A method according to claim 19 wherein step (i) further comprises one, or any combination of the following steps;
i) providing a linking agent and MSH receptor ligand;
ii) providing conditions suitable for linking said agent with MSH receptor ligand; and
iv) bringing the linked molecule in contact with a cell surface to be treated.

21. A method according to claim 19 or claim 20 wherein the linker comprises polyethylene glycol (PEG).

22. A method of preparing a cell culture surface comprising:
i) providing at least one organic monomer;
ii) creating a plasma of said organic monomer; and
iii) coating the surface with said plasma to provide a cell culture surface to which MSH is capable of being associated.

23. A method according to claim 22 when the organic monomer is an amine such as allyl amine, butyl amine, heptyl amine or propyl amine.

24. A method of treatment comprising administering to a patient in need of therapeutic or cosmetic surgery a vehicle of any of claims 1 to 18.
